(19) United States
(12) Patent Application Publication
(10) Pub. No.: US 2023/0108237 A1
(43) Pub. Date: Apr. 6, 2023

(54) Temperature Indicating Label for Perishables

(71) Applicant: Katina Daugherty, St. Louis, MO (US)

(72) Inventor: Katina Daugherty, St. Louis, MO (US)

(21) Appl. No.: 17/959,207

(22) Filed: Oct. 3, 2022

Related U.S. Application Data
(60) Provisional application No. 63/251,555, filed on Oct. 1, 2021.

(51) Int. Cl. G01K 11/165; G01N 33/02

(52) U.S. Cl. CPC G01K 11/165; G01N 33/02

(57) ABSTRACT

A temperature indicating label for perishables includes a protective layer, a thermochromic liquid crystal layer, a silver-plated layer, an adhesive layer, and a release liner. The thermochromic liquid crystal layer is perimetrically superimposed onto the silver-plated layer. The protective layer is perimetrically superimposed onto the thermochromic liquid crystal layer. The protective layer and the silver-plated layer are oppositely positioned of each other about the thermochromic liquid crystal layer as the thermochromic liquid crystal layer displays a color-coded reading to demonstrate the measured temperature of the perishables. The adhesive layer is superimposed onto the silver-plated layer thus allowing the label to be attached to the package or the container of the food. The adhesive layer is oppositely positioned to the thermochromic liquid crystal layer as the release liner is perimetrically attached to the adhesive layer.

The current application claims a priority to the U.S. Provisional Patent application Ser. No. 63/251,555 filed on Oct. 1, 2021. The current application is filed on Oct. 3, 2022, while Oct. 1, 2022 was on a weekend.

### FIELD OF THE INVENTION

The present invention relates generally to a temperature reader to identify the freshness of perishable products. More specifically, the present invention is a temperature indicating label for perishables that reads the temperature of food to measure the freshness of perishable products.

### BACKGROUND OF THE INVENTION

When preparing food for a meal both the freshness of the item needs to be checked and the internal temperature needs to be checked when cooking certain foods to limit the spread of foodborne illnesses. Various grocery stores mark their perishable items with stickers or labels indicating the packing date and a best use by date. Unfortunately, these labels and stickers are only based on when the item was packaged and is simply an estimate on when the food may go bad by and cannot specifically determine when the food itself has gone bad. Additionally, most labels and stickers do not apply for frozen foods as the dates are specifically designed to estimate the shelf life while the product stays frozen.

An objective of the present invention is to provide users with a disposable sticker that measures the temperature of perishables, to help reduce chances of foodborne illnesses from people eating spoiled food. More specifically, the present invention is a temperature indicating label for perishables that can be placed on the package or the container during the initial preparation process, marking the product with a specific color. Further, the present invention may change colors throughout the life of the perishable product, indicating to the user if the food can be used or should be thrown away.

### SUMMARY OF THE INVENTION

The present invention is a temperature indicating label for perishables that changes color to help indicate the freshness of perishable products. The present invention is placed on the package or the container of the perishable food product to determines the accurate reading of the internal temperature. The corresponding temperature reading is displayed to the user through a layer of thermochromic liquid crystal layer of the present invention as a color-coded reading.

### BRIEF DESCRIPTION OF THE DRAWINGS

FIG. 1 is a perspective view of the present invention.

FIG. 2 is a cross-sectional view showing the first embodiment of the present invention.

FIG. 3 is a cross-sectional view showing the second embodiment of the present invention.

FIG. 4 is a top view of the present invention illustrating the white color reading.

FIG. 5 is a top view of the present invention illustrating the red color reading.

FIG. 6 is a top view of the present invention illustrating the reddish orange color reading.

FIG. 7 is a top view of the present invention illustrating the orange color reading.

FIG. 8 is a top view of the present invention illustrating the orangish yellow color reading.

FIG. 9 is a top view of the present invention illustrating the yellow color reading.

FIG. 10 is a top view of the present invention illustrating the green color reading.

FIG. 11 is a top view of the present invention illustrating the yellowish-greenish-blue color reading.

FIG. 12 is a top view of the present invention illustrating the greenish-blue color reading.

FIG. 13 is a top view of the present invention illustrating the blue color reading.

FIG. 14 is a top view of the present invention illustrating the black color reading.

### DETAIL DESCRIPTIONS OF THE INVENTION

All illustrations of the drawings are for the purpose of describing selected versions of the present invention and are not intended to limit the scope of the present invention.

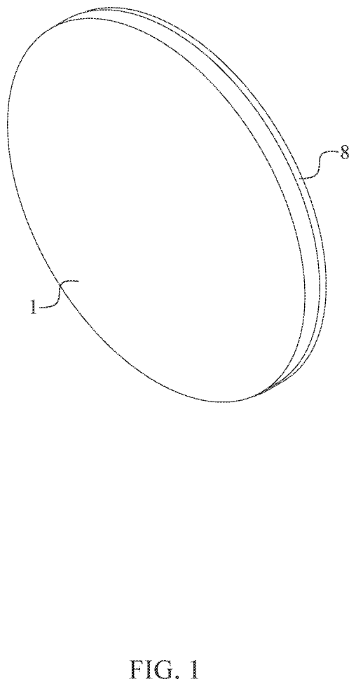

The present invention is a temperature indicating label for perishables that indicates the freshness level of the food. The present invention visually indicates the freshness level or the temperature range of the food so that the consumers can whether the perishable food is safe to consume or not. As shown in FIGS. 1-3, the present invention comprises a protective layer 1, a thermochromic liquid crystal layer 2, a silver-plated layer 3, an adhesive layer 5, and a release line 8. Aforementioned components are integrated into each other so that the present invention can be configured. The present invention is placed/adhered onto the food package or food containers so that the consumers are able to visually identify the freshness level of the food.

In reference to the configuration of the present invention, as shown in FIGS. 1-3, the thermochromic liquid crystal layer 2 that visually display a specific color according to temperature of the food item is perimetrically superimposed onto the silver-plated layer 3. The silver-plated layer 3 provides a flexible and structurally strengthen surface area to protect a bottom surface of the thermochromic liquid crystal layer 2. Furthermore, the silver-plated layer 3 also functions as a thermally conductive material so that the thermochromic liquid crystal layer 2 is able to receive an accurate temperature reading from the food item. The protective layer 1 is perimetrically superimposed onto the thermochromic liquid crystal layer 2 as the protective layer 1 and the silver-plated layer 3 are oppositely positioned of each other about the thermochromic liquid crystal layer 2. In other words, the protective layer 1 shields a top surface of the thermochromic liquid crystal layer 2 from outside element and external temperature variations so that the present invention is able to accurately measure the temperature of the food item. The adhesive layer 5 is superimposed onto the silver-plated layer 3. Furthermore, the adhesive layer 5 and the thermochromic liquid crystal layer 2 are oppositely positioned of each about the silver-plated layer 3 so that the present invention can be adhered to the package or the container of the food item via the adhesive layer 5. The release line 8 is perimetrically attached to the adhesive layer 5 to protect the adhesive properties of the adhesive layer 5 and to prevent activation of the thermochromic liquid crystal layer 2. In other words, when the release layer is attached to the adhesive layer 5, the thermochromic liquid crystal layer 2 is not able to detect any temperature changes via conduction.

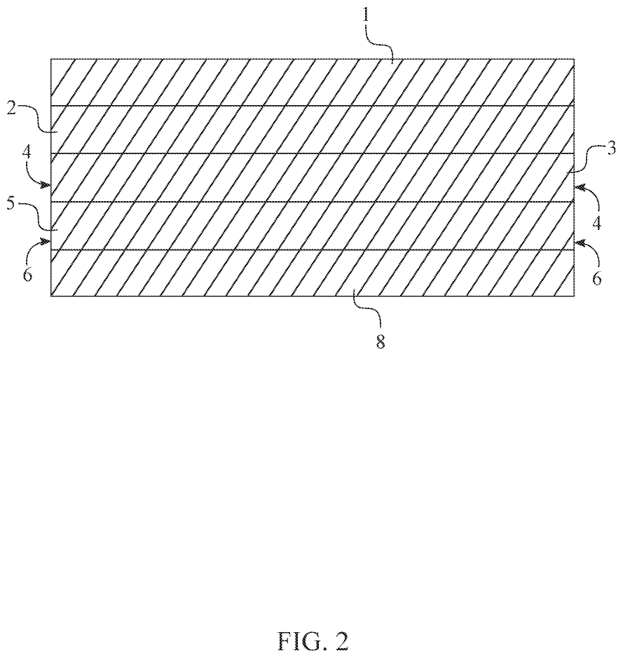

In reference to a first embodiment of the present invention, the adhesive layer 5 comprises a first outer edge 6, and the silver-plated layer 3 comprises a second outer edge 4 as shown in FIG. 1 and FIG. 2. More specifically, the first outer edge 6 is perimetrically positioned to the second outer edge 4 as the adhesive layer 5 fully covers the silver-plated layer 3. In order to prevent activation of the present invention, the adhesive layer 5 is enclosed by the release line 8. For example, when the silver-plated layer 3 is formed into a circular shape, the adhesive layer 5 is formed into a matching circular shape that corresponds with the silver-plated layer 3. Then, the release linear is also formed into a matching circular shape to fully protect the adhesive layer 5.

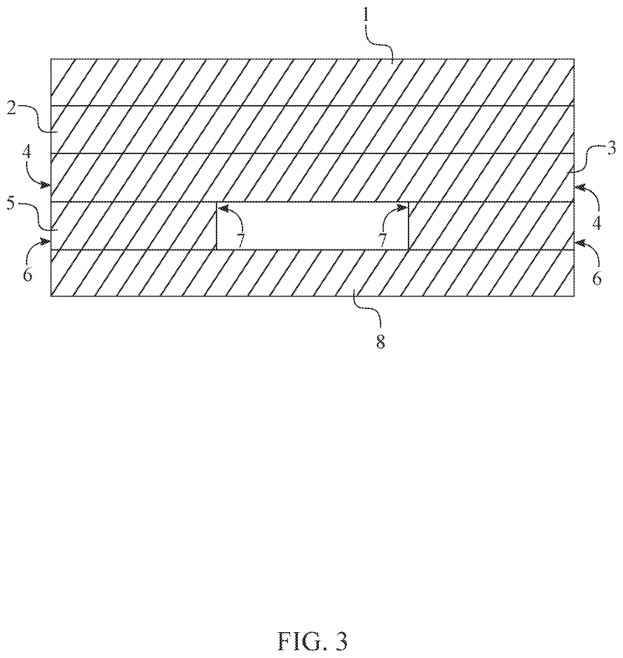

In reference to a second embodiment of the present invention, the adhesive layer 5 comprises the first outer edge 6 and a first inner edge 7, and the silver-plated layer 3 comprises the second outer edge 4 as shown in FIG. 1 and FIG. 3. More specifically, the adhesive layer 5 is formed into a ring-shaped layer as the first inner edge 7 is positioned within the first outer edge 6. The first outer edge 6 is perimetrically positioned to the second outer edge 4, wherein the adhesive layer 5 is inwardly extended from the second outer edge 4. Furthermore, the first inner edge 7 is internally offset from the second outer edge 4 as the adhesive layer 5 partially covers the silver-plated layer 3. In order to prevent activation of the present invention, the adhesive layer 5 and the silver-plated layer 3 are enclosed by the release line 8. For example, when the silver-plated layer 3 is formed into a circular shape, the adhesive layer 5 is formed into a ring shape, wherein an outer diameter of the silver-plated layer 3 is equal to an outer diameter of the adhesive layer 5 thus partially exposing the silver-plated layer 3 within the first inner edge 7. Then, the release linear is also formed into a matching circular shape as the silver-plated layer 3 to fully protect the adhesive layer 5 and the silver-plated layer 3.

In order for the thermochromic liquid crystal layer 2 to visually display the specific color according to temperature of the food item, the protective layer 1 is configured as a translucent layer. As a result, the user can clearly identify the specific color that is displayed through the thermochromic liquid crystal layer 2.

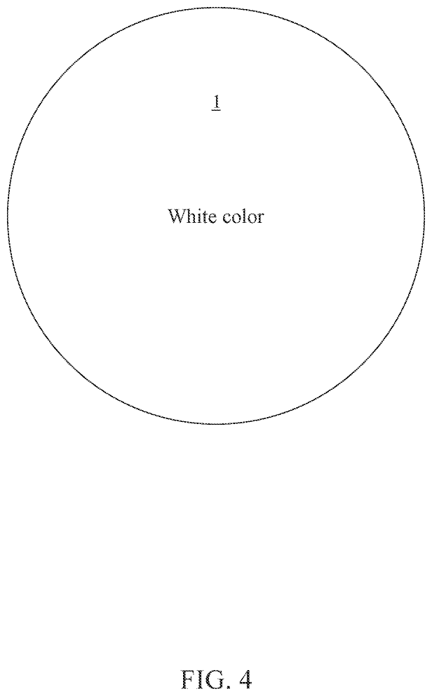

In reference to FIG. 4, the thermochromic liquid crystal layer 2 displays a white color reading for a temperature greater than 165 degrees Fahrenheit. For example, when the present invention is placed on a cooked poultry container, the present invention displays the white color reading to indicate that the poultry item has exceeded the recommended cooking temperature.

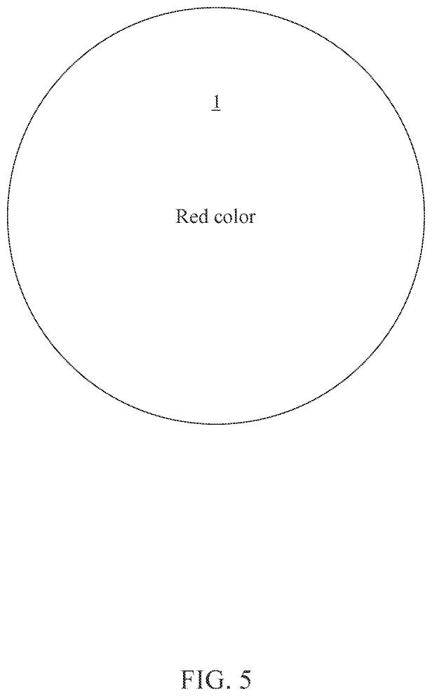

In reference to FIG. 5, the thermochromic liquid crystal layer 2 displays a red color reading for a temperature of 165 degrees Fahrenheit. For example, when the present invention is placed on a cooked poultry container, the present invention displays the red color reading to indicate that the poultry item has reached the recommended cooking temperature.

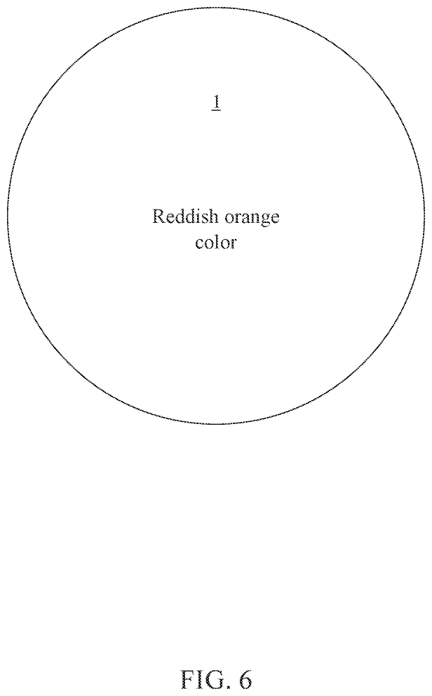

In reference to FIG. 6, the thermochromic liquid crystal layer 2 displays a reddish orange color reading for a temperature range of 165-160 degrees Fahrenheit. For example, when the present invention is placed on a cooked pork container, the present invention displays the reddish orange color reading to indicate that the pork item has exceeded the recommended cooking temperature range.

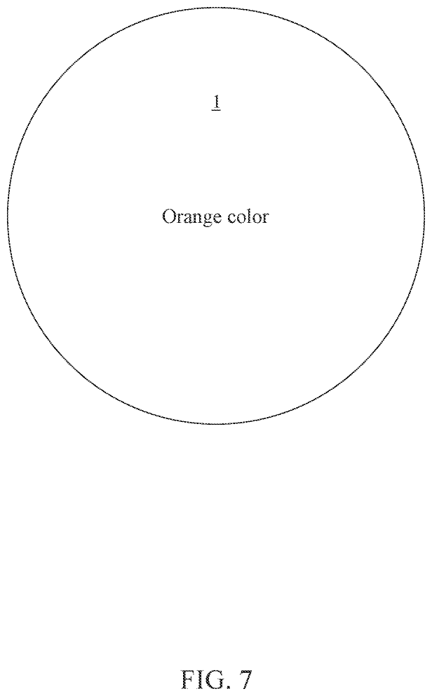

In reference to FIG. 7, the thermochromic liquid crystal layer 2 displays an orange color reading for a temperature range of 160-145 degrees Fahrenheit. For example, when the present invention is placed on a cooked pork container, the present invention displays the orange color reading to indicate that the pork item has reached the recommended cooking temperature range.

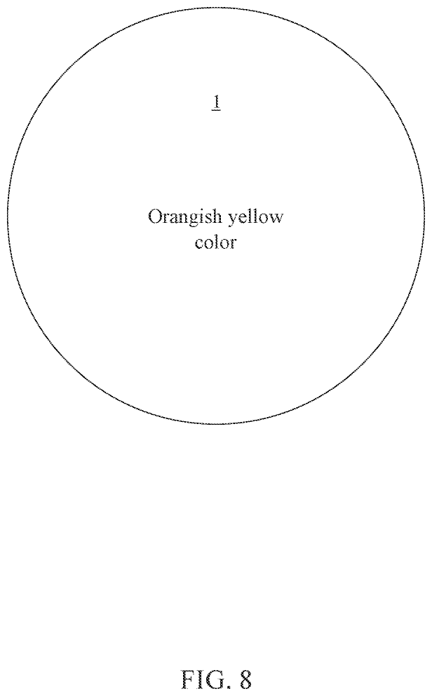

In reference to FIG. 8, the thermochromic liquid crystal layer 2 displays an orangish yellow color reading for a temperature range of 145-140 degrees Fahrenheit. For example, when the present invention is placed on a pre-cooked ham container, the present invention displays the orangish yellow color reading to indicate that the pre-cook ham item has reached the recommended cooking temperature range.

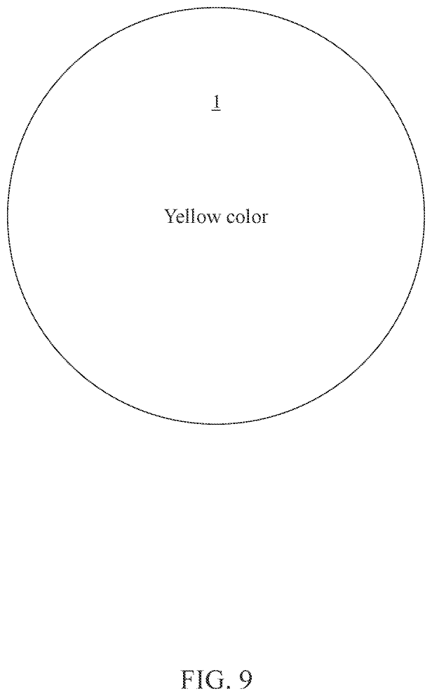

In reference to FIG. 9, the thermochromic liquid crystal layer 2 displays a yellow color reading for a temperature range of 140-100 degrees Fahrenheit. For example, when the present invention is placed on a raw beef container, the present invention displays the yellow color reading to indicate that the raw beef item has reached the spoilage temperature range and is not suitable for cooking.

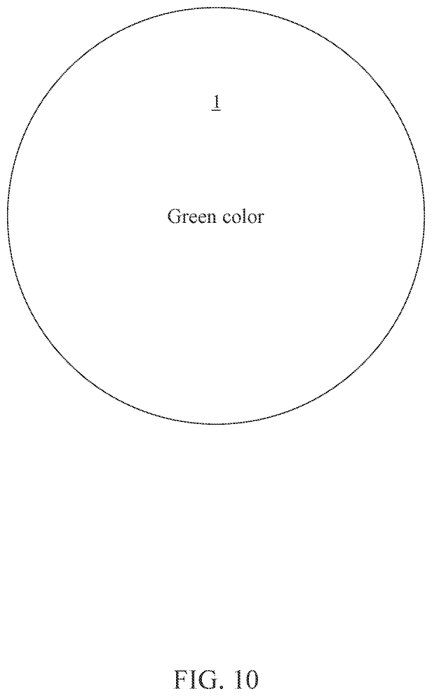

In reference to FIG. 10, the thermochromic liquid crystal layer 2 displays a green color reading for a temperature range of 100-41 degrees Fahrenheit. For example, when the present invention is placed on a raw beef container, the present invention displays the green color reading to indicate that the raw beef item has exceeded the recommended refrigerated temperature.

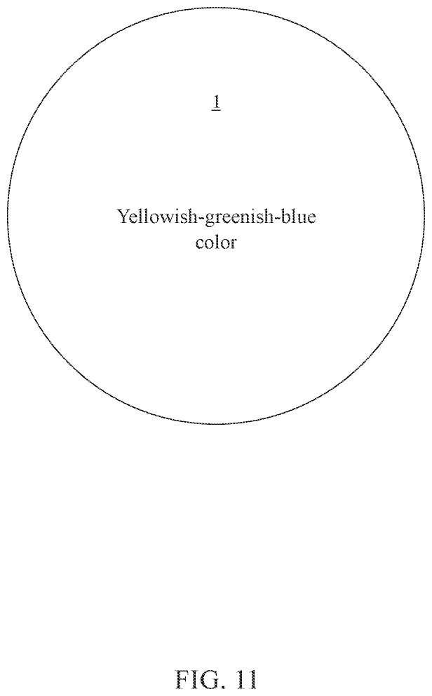

In reference to FIG. 11, the thermochromic liquid crystal layer 2 displays a yellowish-greenish-blue color reading for a temperature of 40 degrees Fahrenheit. For example, when the present invention is placed on a raw beef container, the present invention displays the yellowish-greenish-blue color reading to indicate that the raw beef item is within the recommended refrigerated temperature.

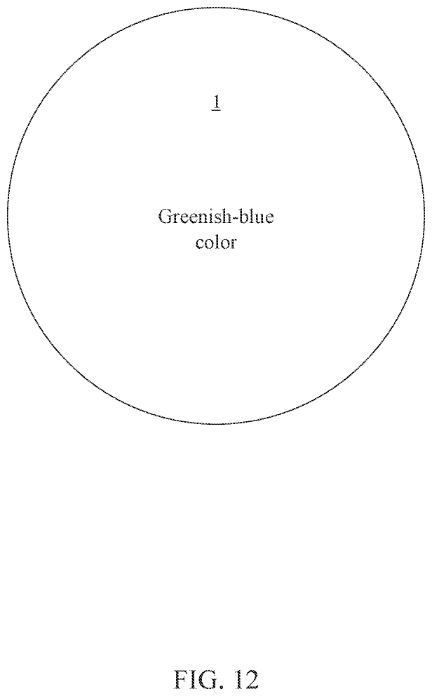

In reference to FIG. 12, the thermochromic liquid crystal layer 2 displays a greenish-blue color reading for a temperature range of 40-1 degrees Fahrenheit. For example, when the present invention is placed on a frozen beef container, the present invention displays the greenish-blue color reading to indicate that the frozen beef item has exceeded the recommended storage temperature.

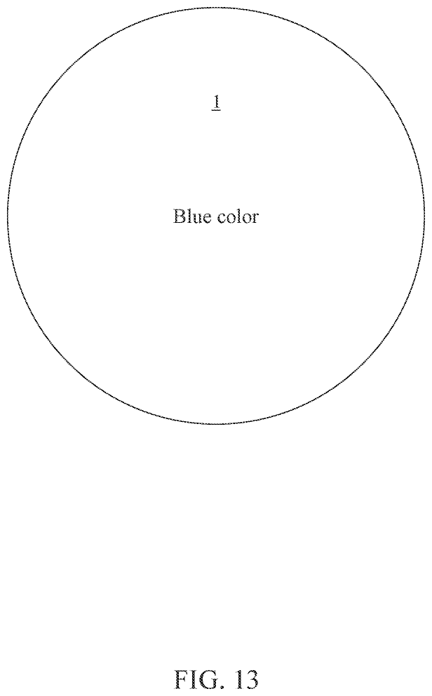

In reference to FIG. 13, the thermochromic liquid crystal layer 2 displays a blue color reading for a temperature of 0 degrees Fahrenheit. For example, when the present invention is placed on a frozen beef container, the present invention displays the blue color reading to indicate that the frozen beef item is within the recommended storage temperature.

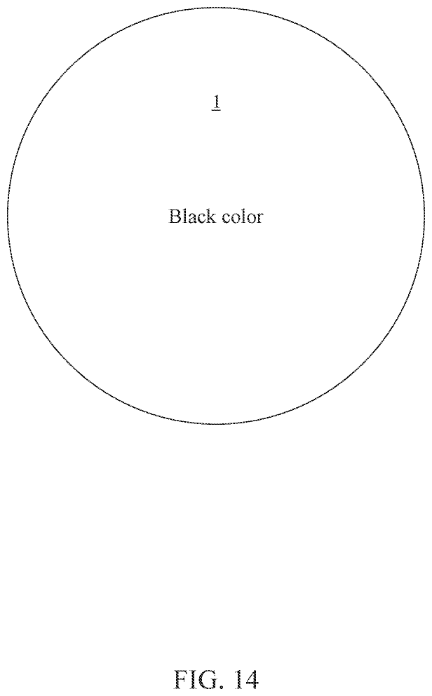

In reference to FIG. 14, the thermochromic liquid crystal layer 2 displays a black color reading to show at least one defective circumstances of the present invention. In other words, when the present invention displays the black color reading, the present invention is considered as a defective embodiment or expired embodiment wherein the thermochromic liquid crystal layer 2 is not functional. For examples, the present invention displays the black color reading when the label is activated more than 60 days and the temperature ranges between 138-144 degrees Fahrenheit. The present invention also displays the black color reading when the label is removed from the original package. Furthermore, the present invention displays a purple color reading when the label is activated more than 60 days and the temperature drops below 138 degrees Fahrenheit.

In an alternative embodiment, the present invention can be configured into a digital apparatus. The digital apparatus can be designed with a touch screen or with analog push buttons. The digital apparatus is designed to provide the user with a screen to display the following: color status, date, product, temperature, a wireless unit indicator, time, timer, power (on/off button), speaker, mic, and camera. The digital apparatus can be powered by a solar power, an AC/DC power or a battery. The digital apparatus can also include a QR code that allows the user to connect it to their smart phones and a USB interface to connect it to a computer and a reset button. When the digital apparatus functions similar to a monitoring device, the digital apparatus can be attached to the food item by a belt loop, or it can be applied to the food item directly and secured with a clear wrap material. When the digital apparatus functions similar to a prob, the digital apparatus can be in contact with the food item and stored with the package or the container. When the digital apparatus can also be built into a container or a package wherein the corresponding container or the package internally contain the food item.

Although the invention has been explained in relation to its preferred embodiment, it is to be understood that many other possible modifications and variations can be made without departing from the spirit and scope of the invention as hereinafter claimed.

What is claimed is:

1. A temperature indicating label for perishables comprising:
a protective layer;
a thermochromic liquid crystal layer;
a silver-plated layer;
an adhesive layer;
a release liner;
the thermochromic liquid crystal layer being perimetrically superimposed onto the silver-plated layer;
the protective layer being perimetrically superimposed onto the thermochromic liquid crystal layer;
the protective layer and the silver-plated layer being oppositely positioned of each other about the thermochromic liquid crystal layer;
the adhesive layer being superimposed onto the silver-plated layer;
the adhesive layer and the thermochromic liquid crystal layer being oppositely positioned of each other about the silver-plated layer; and
the release liner being perimetrically attached to the adhesive layer.

2. The temperature indicating label for perishables as claimed in claim 1 comprising:
the adhesive layer comprising a first outer edge;
the silver-plated layer comprising a second outer edge;
the first outer edge being perimetrically positioned to the second outer edge; and
the adhesive layer being enclosed by the release liner.

3. The temperature indicating label for perishables as claimed in claim 1 comprising:
the adhesive layer comprising a first outer edge and a first inner edge;
the silver-plated layer comprising a second outer edge;
the first inner edge being positioned within the first outer edge;
the first outer edge being perimetrically positioned to the second outer edge;
the first inner edge being internally offset from the second outer edge; and
the adhesive layer and the silver-plated layer being enclosed by the release liner.

4. The temperature indicating label for perishables as claimed in claim 1, wherein the protective layer is a translucent layer.

5. The temperature indicating label for perishables as claimed in claim 1, wherein the thermochromic liquid crystal layer displays a white color reading for a temperature greater than 165 degrees Fahrenheit.

6. The temperature indicating label for perishables as claimed in claim 1, wherein the thermochromic liquid crystal layer displays a red color reading for a temperature of 165 degrees Fahrenheit.

7. The temperature indicating label for perishables as claimed in claim 1, wherein the thermochromic liquid crystal layer displays a reddish orange color reading for a temperature range of 165-160 degrees Fahrenheit.

8. The temperature indicating label for perishables as claimed in claim 1, wherein the thermochromic liquid crystal layer displays an orange color reading for a temperature range of 160-145 degrees Fahrenheit.

9. The temperature indicating label for perishables as claimed in claim 1, wherein the thermochromic liquid crystal layer displays an orangish yellow color reading for a temperature range of 145-140 degrees Fahrenheit.

10. The temperature indicating label for perishables as claimed in claim 1, wherein the thermochromic liquid crystal layer displays a yellow color reading for a temperature range of 140-100 degrees Fahrenheit.

11. The temperature indicating label for perishables as claimed in claim 1, wherein the thermochromic liquid crystal layer displays a green color reading for a temperature range of 100-41 degrees Fahrenheit.

12. The temperature indicating label for perishables as claimed in claim 1, wherein the thermochromic liquid crystal layer displays a yellowish-greenish-blue color reading for a temperature of 40 degrees Fahrenheit.

13. The temperature indicating label for perishables as claimed in claim 1, wherein the thermochromic liquid crystal layer displays a greenish-blue color reading for a temperature of 40-1 degrees Fahrenheit.

14. The temperature indicating label for perishables as claimed in claim 1, wherein the thermochromic liquid crystal layer displays a blue color reading for a temperature of 0 degrees Fahrenheit.

15. The temperature indicating label for perishables as claimed in claim 1, wherein the thermochromic liquid crystal layer displays a black color reading to show at least one defective circumstances.
